(19) United States
(12) Patent Application Publication
(10) Pub. No.: US 2009/0100558 A1
(43) Pub. Date: Apr. 23, 2009

(54) Beauty Tape

(76) Inventor: Louis Benjamin Smith, Detroit, MI (US)

Correspondence Address: L. BENJAMIN SMITH, P.O. BOX 47591, OAK PARK, MI, 48237, US

(21) Appl. No.: 11/868,516

(22) Filed: Oct. 7, 2007

(51) Int. Cl. A41D 13/00; A45D 44/12; A61F 13/00

(52) U.S. Cl. 2/9; 2/468; 2/174; 602/41

(57) ABSTRACT

Beauty Tape is a flexible BAND-AID type material cut and shape to protect the forehead, temples, neck and ears from accidental burns by hot utensils during the toilet process. The tape would not contain the medicated center found in BAND-AID type bandages. BEAUTY TAPE can be made from the same conforming materials currently marketed by the makers CURAD and BAND-AID. The strips of tapes can be contoured and made easy to apply to the desired area. The strips of tapes can be used one at a time or uniformly to form a preventative layer of tape on the head, neck and ears.

### BACKGROUND OF THE INVENTION

1. Field of Invention

The present invention relates to an applicable material use to prevent burns during the pressing and curling of hair with hot utensils.

2. Discussion of Background

Hot utensils are commonly used to press and curl female hair. Research has verified that most females either have routine hair appointments or have their hair styled for an occasion. During this process often times accidents occur and the results are degree burns about the head and other areas. These burns occur primarily in five locations; the fore head, the temples, the neck, the ears and the scalp. These burns are not only painful but very unsightly and depending upon the occasion and the timing will ruin a female's day. Four of the five areas where a woman can be burned are highly visible. And although a scalp burn is very painful in all but a very few cases the burn can be hidden by hair and will go unnoticed. To a woman there is nothing worst than to experience an accidental burn. Beyond the pain there is the emotional trauma associated with an accident of this type. After witnessing a burn of my wife's neck I came upon a preventive measure that was simple yet effective. I place a large BAND-AID on her forehead and applied a hot curling iron to the area. She exhibited the reflexive response of the hot utensil being placed on her forehead and although she could sense temperature the resulting contact did not leave a burn.

That simple experiment confirmed that a BAND-AID type material would be ideal for this application.

### SUMMARY OF THE INVENTION

Beauty Tape will provide protection against accidental burns by hot utensils to the ears, forehead, temple and neck. BEAUTY TAPE can be made utilizing existing materials currently on the market. BEAUTY TAPE can be marketed in a general size and further trim to fit smaller sizes. BEAUTY TAPE may be constructed generally from existing fabrics that are typically used to make BAND-AID and CURAD type bandages.

### BRIEF DESCRIPTION OF DRAWINGS

In the drawings,

FIG. 1 is a profile displaying the use of Beauty Tape in concert.

FIG. 2 is a rendering of a forehead tape.

FIG. 3 is a rendering of a temple tape.

FIG. 4 is a rendering of a tape for the ear.

FIG. 5 is a rendering of a rear neck tape.

FIG. 6 is a rendering of what a side neck tape might be.

What appear to be drawings on p. 4 and p. 5 are photos converted to sketches for clarity.

These photos/sketches retain their original labels.

Photo 1 depicts forehead application of Beauty Tape.

Photo 2 depicts forehead and temple application.

Photo 3 depicts a profile of the temple, upper and lower neck application.

Photo 4 depicts another profile of the upper and lower neck application.

Photo 5 depicts the rear lower neck application.

Photo 6 depicts a right profile of Beauty Tape.

### DESCRIPTION OF THE PREFERRED EMBODIMENT

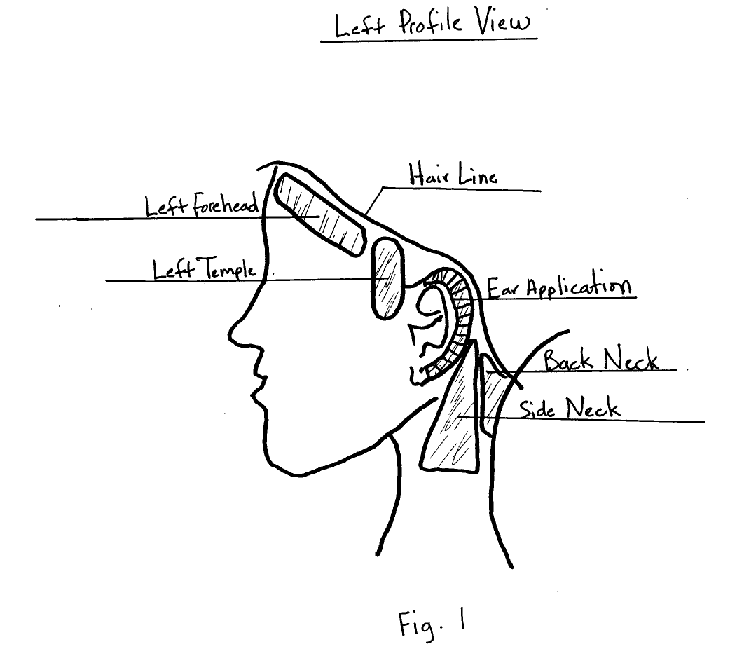

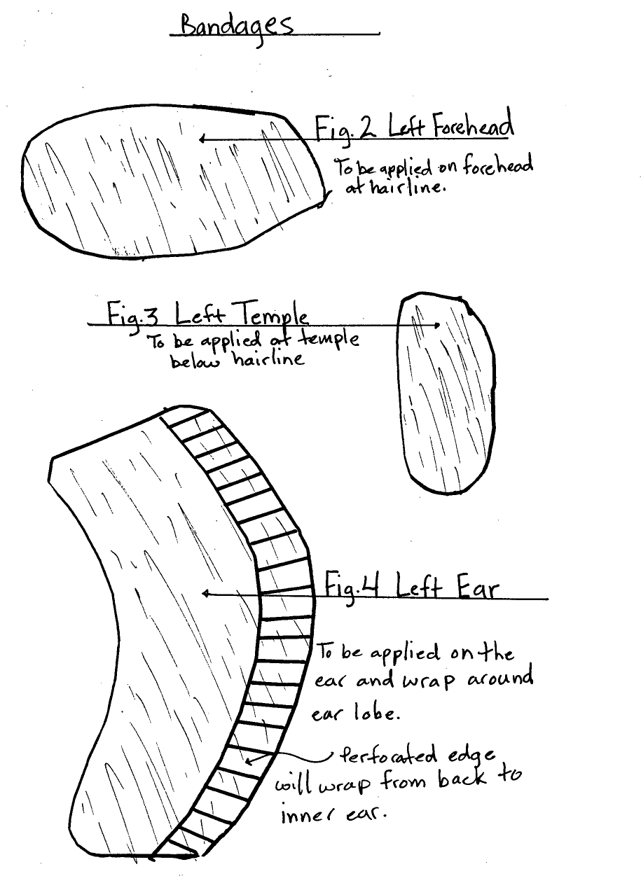

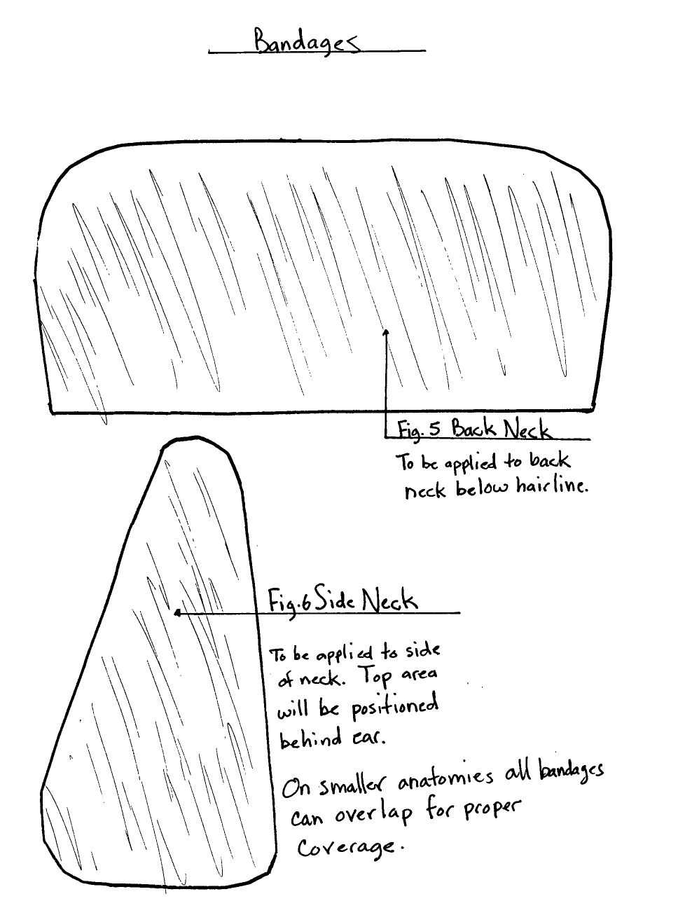

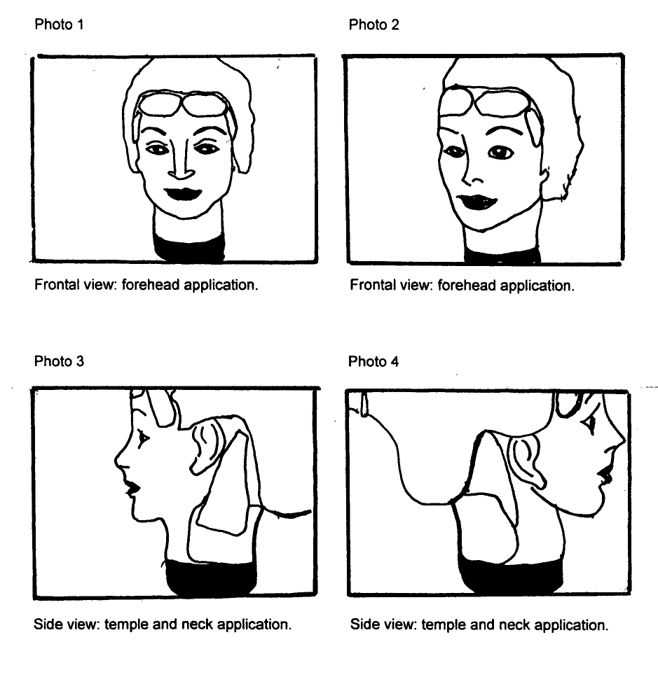

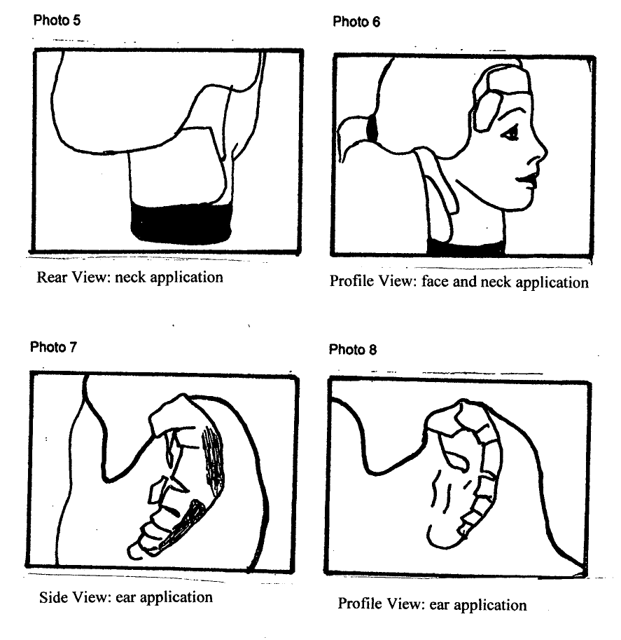

FIG. 1 details the probable alignment of the individual patches and the approximate location on the head and neck area. The fact that no two anatomies are alike a general size would be market with an ability to be trimmed and adjusted to fit. The basic design and composition could be likened to materials used in commercially available bandages already on the market. The shape of the forehead tape shown in FIG. 2 may be approximately 2¾ inches long and approximately 1⅜ inches high with rounded ends with a slight indent on the outer lower edge to accommodate the top portion of the temple tape (FIG. 3) and account for the curvature of the forehead. Shown in FIG. 3 the temple tape is approximately 1¾ inches high and approximately one inch wide. The tape for the ear may be approximately 4 inches high and 2 inches wide. The shape is that of a large bracket symbol with approximately ½ to ¾ inch precut forming flaps that can wrap the edge of the ear. This application will offer protection to the back and edge of the ear from burns. FIG. 5 is the neck tape. It is approximately 6¾ inches long and 2½ inches high and offers protection to the rear neck. The triangular shape tape in FIG. 6 is for the side neck and behind the ear. The dimension is approximately 4 inches high, ¾ inch at the top and 2 inches at the bottom. One side of the tape would be layered with a mild adhesive with the opposite side consisting of conforming flexible impermeable material and able to adhere to dry skin.

It is important that the proper consistency is attained for the adhesive. If the adhesive is too strong the tape will pull at hair follicles along the hair line. Too much adhesive and the application become messy and counter productive. On the other hand too little adhesive and the tape will not hold its position for the duration of the styling process. For home use it is not unlikely that the same tape may be applied more than once. In the case of salon use the tape would be discarded after a single use.

The tape (all the necessary pieces to protect the entire forehead, temples, ears, and neck) may be individually packaged in a small container for marketing, shipping and use. One key application requirement is that BEAUTY TAPE must not impede the styling process and that it abuts the hair line for maximum skin protection.

The invention claims are:

1. A burn preventer claim: wherein flexible fabric patches are placed on the head, ears and neck to prevent burns to these areas by hot styling utensils. These fabric patches may be cut and shape to fit any size anatomy.

2. A burn preventer claim: wherein materials currently on the market may be utilized to manufacture Beauty Tape.

3. A burn preventer claim: wherein fabric patches would allow some passage of heat.

4. A burn preventer claim: wherein bandage materials would contain a mild adhesive to facilitate application and removal.
